(19) United States
(12) Patent Application Publication
(10) Pub. No.: US 2006/0067538 A1
(43) Pub. Date: Mar. 30, 2006

(54) Acoustic device with a noise reduction unit

(76) Inventor: Dawei Huang, Kaohsiung City (TW)

Correspondence Address: MCNEES, WALLACE & NURICK LLC, 100 PINE STREET, P.O. BOX 1166, HARRISBURG, PA, 17108-1166, US

(21) Appl. No.: 11/068,578

(22) Filed: Feb. 28, 2005

(30) Foreign Application Priority Data
Aug. 17, 2004 (CN) 200420082879.0

(51) Int. Cl. A61F 11/06

(52) U.S. Cl. 381/71.1

(57) ABSTRACT

An acoustic device includes a resonance chamber and a noise reduction unit. The noise reduction unit serves to combat noise in the resonance chamber, and includes first and second electro-acoustic transducers, a controller, and a sound-absorbing member. The first electro-acoustic transducer generates a first electrical signal that corresponds to the noise in the resonance chamber. The controller generates a second electrical signal. The second electro-acoustic transducer generates a sound wave that corresponds to the second electrical signal and that counteracts a portion of the noise in the resonance chamber. The sound-absorbing member includes a porous body that is mounted in that resonance chamber and that absorbs another portion of the noise in the resonance chamber.

### CROSS-REFERENCE TO RELATED APPLICATION

This application claims priority of Chinese application no. 200420082879.0, filed on Aug. 17, 2004.

### BACKGROUND OF THE INVENTION

1. Field of the Invention

The invention relates to an acoustic device, more particularly to an acoustic device that includes a noise reduction unit.

2. Description of the Related Art

A conventional acoustic device, such as those found in headphones, includes a resonance chamber, a speaker, and a noise-reduction circuit. The speaker is mounted in the resonance chamber. The noise-reduction circuit is operatively associated with the speaker for reducing noise in the resonance chamber.

The aforementioned conventional acoustic device is disadvantageous in that its noise reduction circuit, due to circuit delay, is unable to effectively combat noise in the resonance chamber.

### SUMMARY OF THE INVENTION

Therefore, the object of the present invention is to provide an acoustic device that can overcome the aforesaid drawback of the prior art.

According to the present invention, an acoustic device comprises a resonance chamber and a noise reduction unit. The noise reduction unit serves to combat noise in the resonance chamber, and includes first and second electro-acoustic transducers, a controller, and a sound-absorbing member. The first electro-acoustic transducer is mounted in the resonance chamber, and is operable so as to generate a first electrical signal that corresponds to the noise in the resonance chamber. The controller is coupled to the first electro-acoustic transducer, and is operable so as to receive the first electrical signal generated by the first electro-acoustic transducer and so as to generate a second electrical signal. The second electro-acoustic transducer is mounted in the resonance chamber, is coupled to the controller, and is operable so as to generate a sound wave that corresponds to the second electrical signal and that counteracts a portion of the noise in the resonance chamber. The sound-absorbing member includes a porous body that is mounted in the resonance chamber and that absorbs another portion of the noise in the resonance chamber.

### BRIEF DESCRIPTION OF THE DRAWINGS

Other features and advantages of the present invention will become apparent in the following detailed description of the preferred embodiment with reference to the accompanying drawings, of which:

FIG. 1 is an exploded perspective view of the preferred embodiment of an acoustic device according to the present invention;

FIG. 2 is partly sectional view of the preferred embodiment in an assembled state;

FIG. 3 is a schematic circuit block diagram of the preferred embodiment;

FIG. 4 is a perspective view of the first preferred embodiment applied in a pair of headphones;

FIG. 5 is a schematic circuit diagram to illustrate a pair of controller circuits of the preferred embodiment; and

FIG. 6 is a graph illustrating exemplary frequency responses of the conventional acoustic device and the preferred embodiment.

### DETAILED DESCRIPTION OF THE PREFERRED EMBODIMENT

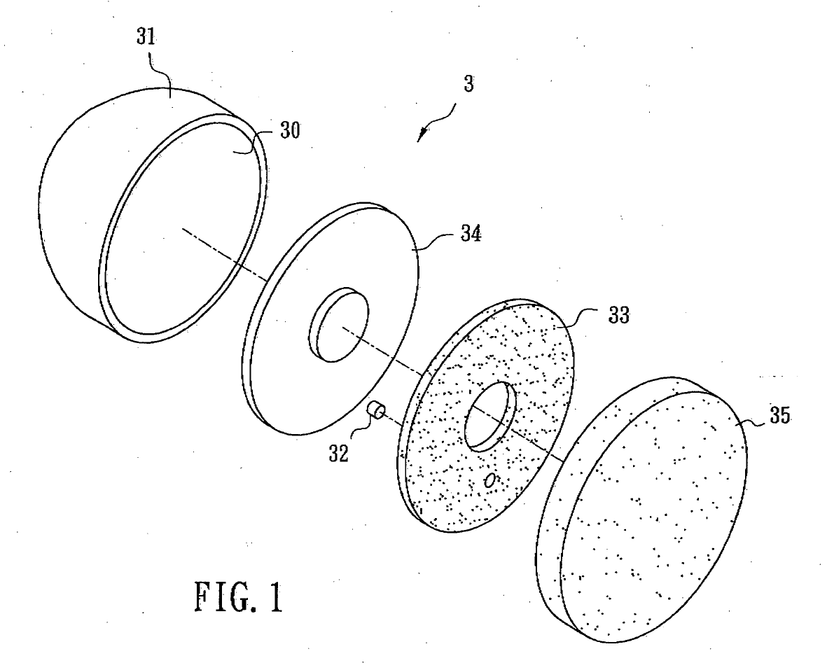

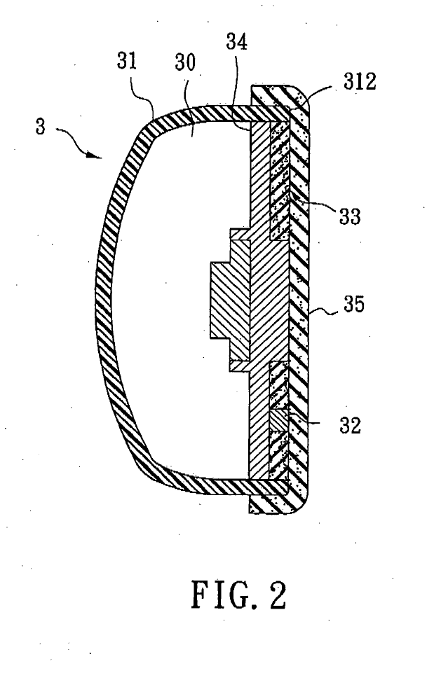

Referring to FIGS. 1 to 3, the preferred embodiment of an acoustic device 3 according to this invention is shown to include a resonance chamber 31, first and second electro-acoustic transducers 32, 34, a controller circuit 2, and a sound-absorbing member.

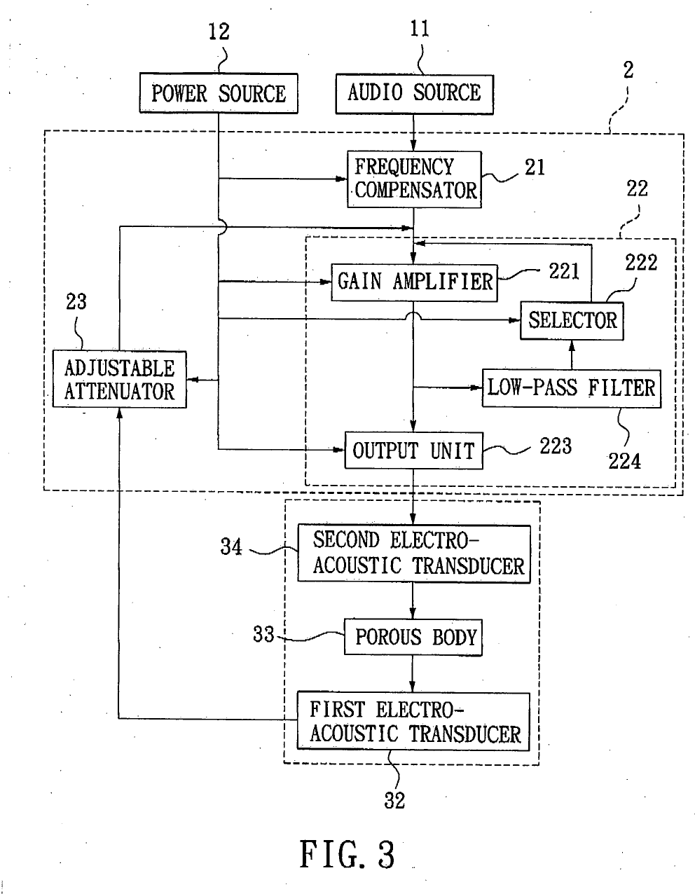

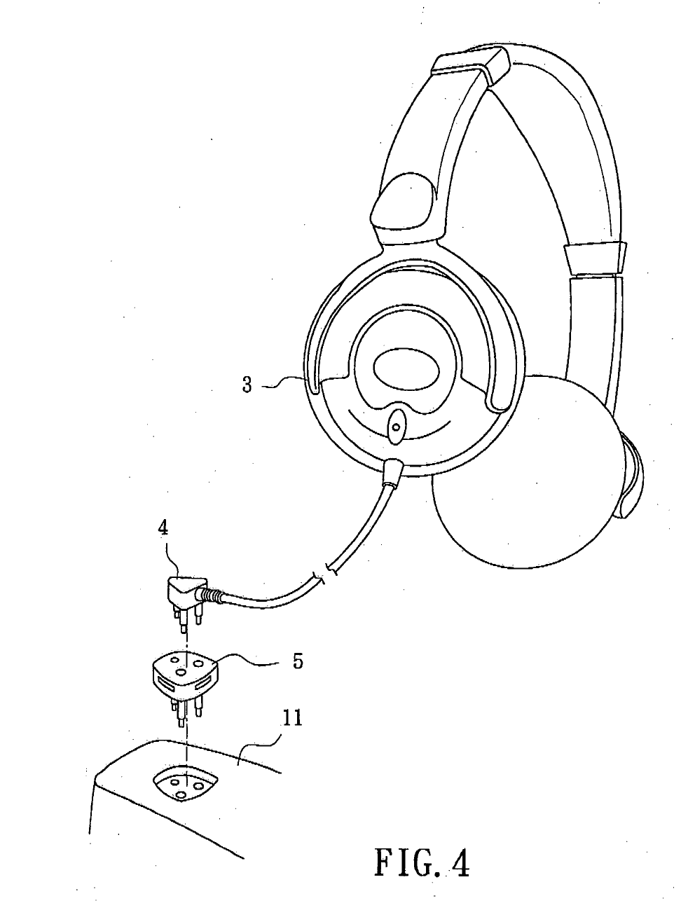

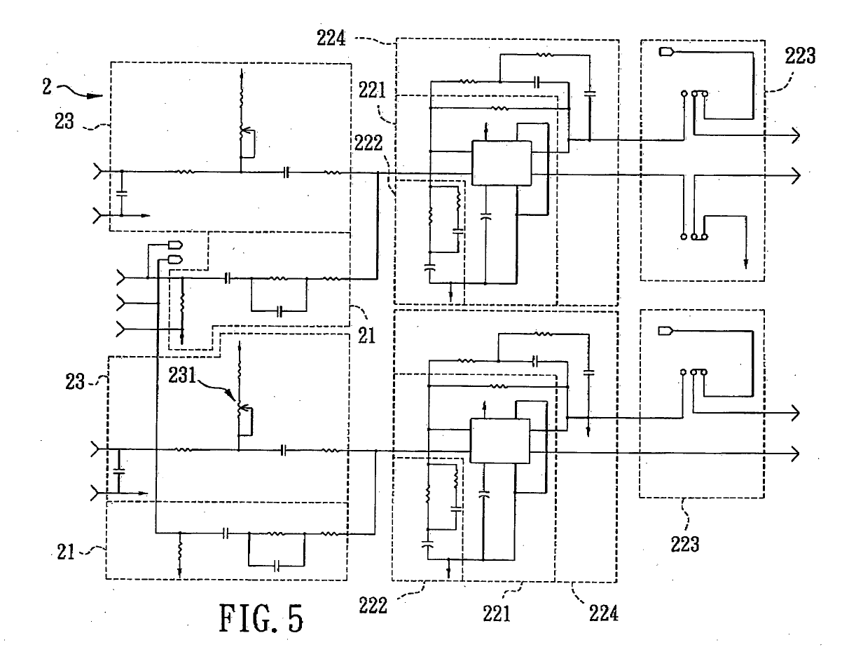

The acoustic device 3, in this embodiment, is applied to a pair of headphones, as illustrated in FIG. 4. The headphones include a pair of the acoustic devices 3, a headphone connector 4, and a headphone adapter 5. The acoustic devices 3 of the headphones are disposed on both sides of the headphones, and are coupled to each other, as best shown in FIG. 5. Since the acoustic devices 3 on either side of the headphones are the same in construction and operation, only one will be described. For clarity, FIG. 3 shows only one of the acoustic devices 3.

The first and second electro-acoustic transducers 32, 34, the controller circuit 2, and the sound-absorbing member constitute a noise reduction unit for combating noise, such as unwanted resonances, in the resonance chamber 31. It is noted that the noise in the resonance chamber 31 includes low frequency components, which are 100 Hz and below, and high frequency components, which are 2 KHz and above.

The first electro-acoustic transducer 32 is mounted in the resonance chamber 31. In this embodiment, the first electro-acoustic transducer 32 is a microphone.

The controller circuit 2 is mounted in the resonance chamber 31, and includes a frequency compensator 21, a mixer 22, and an adjustable attenuator 23. The mixer 22 of the controller circuit 2 includes a gain amplifier 221, a selector 222, a low-pass filter 224, and an output unit 223. The frequency compensator 21 of the controller circuit 2 has an input side adapted to be coupled to an audio source 11, and an output side. The gain amplifier 221 of the mixer 22 of the controller circuit 2 has an input side coupled to the output side of the frequency compensator 21 of the controller circuit 2, and an output side. The selector 222 of the mixer 22 of the controller circuit 2 has an input side, and an output side coupled to the input side of the gain amplifier 221 of the mixer 22 of the controller circuit 2. In this embodiment, the selector 222 of the mixer 22 of the controller circuit 2 has an asymmetrical RC filter structure. The low-pass filter 224 of the mixer 22 of the controller circuit 2 has an input side coupled to the output side of the gain amplifier 221 of the mixer 22 of the controller circuit 2, and an output side that is coupled to the input side of the selector 222 of the mixer 22 of the controller circuit 2. The output unit 223 of the mixer 22 of the controller circuit 2 has an input side coupled to the output side of the gain amplifier 221 of the mixer 22 of the controller circuit 2, and an output side. The adjustable attenuator 23 of the controller circuit 2 has an input side coupled to the first electro-acoustic transducer 32, and an output side that is coupled to the input side of the gain amplifier 221 of the mixer 22 of the controller circuit 2. In this embodiment, the adjustable attenuator 23 of the controller circuit 2 includes a variable resistor.

In an alternative embodiment, the controller circuit 2 is mounted in the headphone adapter 5 of the headphones.

The frequency compensator 21, the mixer 22, and the adjustable attenuator 23 of the controller circuit 2 are adapted to be coupled to an external power source 12.

The second electro-acoustic transducer 34 is coupled to the output side of the output unit 223 of the mixer 22 of the controller circuit 2. In this embodiment, the second electro-acoustic transducer 34 is a speaker.

In operation, the first electro-acoustic transducer 32 generates a first electrical signal that corresponds to the low frequency components of the noise in the resonance chamber 31. The adjustable attenuator 23 of the controller circuit 2 generates a second electrical signal that has a phase opposite to that of the first electrical signal generated by the first electro-acoustic transducer 32. The frequency compensator 21 of the controller circuit 2 generates a third electrical signal that corresponds to an audio signal generated by the audio source 11. The gain is amplifier 221 of the mixer 22 of the controller circuit 2 generates an output signal that corresponds to the combination of the second electrical signal generated by the adjustable attenuator 23 of the controller circuit 2 and the third electrical signal generated by the frequency compensator 21 of the controller circuit 2. The output unit 223 of the mixer 22 of the controller circuit 2 provides the output signal generated by the gain amplifier 221 of the mixer 22 of the controller circuit 2 to the second electro-acoustic transducer 34. The second electro-acoustic transducer 34 generates a first sound wave that counteracts the low frequency components of the noise in the resonance chamber 31, and a second sound wave that corresponds to the third electrical signal. The low-pass filter 224 of the mixer 22 of the controller circuit 2 filters the output signal generated by the gain amplifier 221 of the mixer 22 of the controller circuit 2 prior to receipt by the selector 222 of the mixer 22 of the controller circuit 2.

The sound-absorbing member includes a porous body 33 that is mounted in the resonance chamber 31 and that absorbs the high frequency components of the noise in the resonance chamber 31. In this embodiment, the porous body 33 of the sound-absorbing member is made from a polyurethane (PU) foam material. It is noted that the porous body 33 of the sound-absorbing member is compressed such that the thickness ratio between the initial thickness and the compressed thickness ranges between 3:1 and 40:1, preferably between 3;1 and 30:1. For example, a PU foam material with an initial thickness of 20 mm may be compressed to 6 mm.

It is noted that the first electro-acoustic transducer 32 may be disposed anywhere in the resonance chamber 31. Preferably, the first electro-acoustic transducer 32 is disposed in front of the second electro-acoustic transducer 34 and is embedded within the porous body 33 of the sound-absorbing member.

The resonance chamber 31 has an opening 30. The acoustic device 3 further includes a protective cover 35 that covers the opening 30 of the resonance chamber 31 and that serves to prevent dust particles from entering the resonance chamber 31.

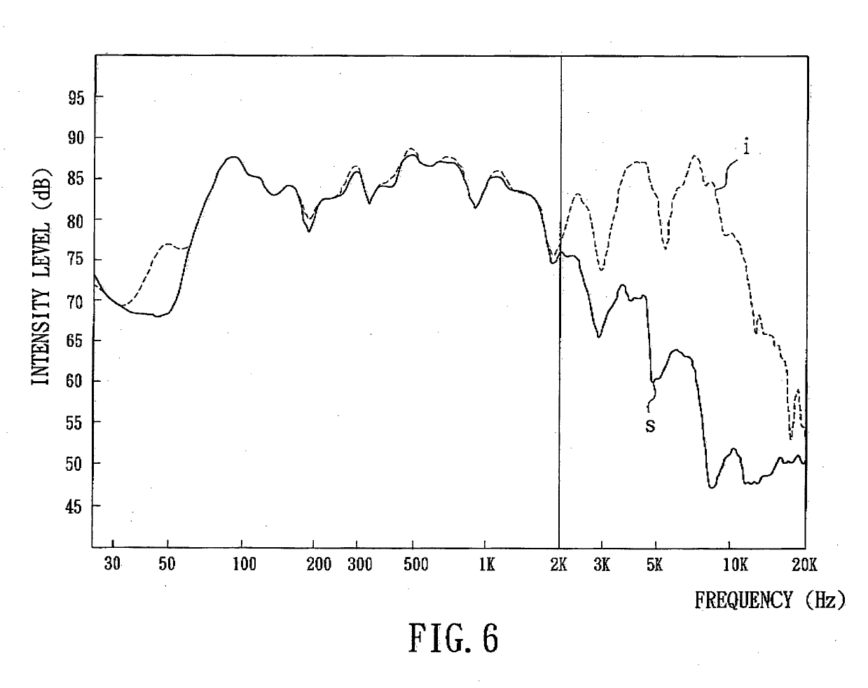

From experimental results, with further reference to FIG. 6, both the conventional acoustic device and the acoustic device 3 of this invention were tested. It is evident from the graph that the intensity level of the high frequency components of the noise in the resonance chamber 31 of the acoustic device 3 of this invention, as indicated by the solid line (s), is substantially lower than that of the conventional acoustic device, as indicated by the imaginary line (i) Hence, it is confirmed that the application of the porous body 33 of the sound-absorbing member does indeed reduce the high frequency components of the noise in the resonance chamber 31 of the acoustic device 3.

While the present invention has been described in connection with what is considered the most practical and preferred embodiment it is understood that this invention is not limited to the disclosed embodiment but is intended to cover various arrangements included within the spirit and scope of the broadest interpretation so as to encompass all such modifications and equivalent arrangements.

1. An acoustic device, comprising:
a resonance chamber; and
a noise reduction unit for combating noise in said resonance chamber, said noise reduction unit including
a first electro-acoustic transducer mounted in said resonance chamber, and operable so as to generate a first electrical signal that corresponds to the noise in said resonance chamber,
a controller coupled to said first electro-acoustic transducer, and operable so as to receive the first electrical signal generated by said first electro-acoustic transducer, and so as to generate a second electrical signal,
a second electro-acoustic transducer mounted in said resonance chamber, coupled to said controller, and operable so as to generate a sound wave that corresponds to the second electrical signal and that counteracts a portion of the noise in said resonance chamber, and
a sound-absorbing member including a porous body that is mounted in said resonance chamber and that absorbs another portion of the noise in said resonance chamber.

2. The acoustic device as claimed in claim 1, wherein said porous body is made from a polyurethane foam material.
